(19) United States
(12) Patent Application Publication
(10) Pub. No.: US 2023/0310473 A1
(43) Pub. Date: Oct. 5, 2023

(54) METHOD FOR TREATING OBESITY AND/OR HYPERTENSION

(71) Applicants: THE ROYAL INSTITUTION FOR THE ADVANCEMENT OF LEARNING/MCGILL UNIVERSITY, Montréal (CA); UNIVERSITY OF TENNESSEE RESEARCH FOUNDATION, Memphis, TN (US)

(72) Inventors: Salwa KARBOUNE, Dollard des Ormeaux (CA); Feng TIAN, Montréal (CA); Andrea HILL, Dorval (CA); Amal SAHYOUN, Saint-Laurent (CA); Modar KASSAN, Memphis, TN (US); Young In KWON, Seoul (KR); Undral MUNKHSAIKHAN, Lakeland, TN (US); Mehdi SIROUSPOUR, L'Ile-Bizard (CA)

(21) Appl. No.: 18/194,374

(22) Filed: Mar. 31, 2023

Related U.S. Application Data
(60) Provisional application No. 63/326,567, filed on Apr. 1, 2022.

(51) Int. Cl. A61K 31/702; A61P 3/04; A61P 9/12

(52) U.S. Cl. CPC A61K 31/702; A61P 3/04; A61P 9/12

(57) ABSTRACT

There is provided a method of treating obesity and/or hypertension in a subject in need thereof by administering a therapeutically effective amount of a low molecular weight levan or a pharmaceutical composition comprising the low molecular weight levan and a pharmaceutically acceptable excipient.

### CROSS REFERENCE TO A RELATED APPLICATION

The present application claims priority from U.S. Provisional Patent Application 63/326,567 filed on Apr. 1, 2022 and herewith incorporated by reference in its entirety.

### FEDERAL FUNDING

This invention was made with government support under federal grant number 7R01HL150360-02 awarded by the National Institutes of Health. The government has certain rights in the invention.

### TECHNICAL FIELD

This disclosure relates to the field of polysaccharides such as levans, methods and uses thereof for improving the health of a subject in need thereof.

### BACKGROUND OF THE ART

Fructan polysaccharides, fructooligomers and fructooligosaccharides (FOSs) or oligofructoses (OFSs) with different degrees of polymerization have been demonstrated to have health benefits as prebiotics. They are divided into three subgroups depending on the type of glycosidic bonds: (i) inulin series, (ii) neo series, and (iii) levan series. Inulins have β (2→1) glycosidic bonds often with a terminal glucose, levans have β (2→6) glycosidic linkages, while neo-OFSs can have both types of glycosidic linkages (i.e. the β (2→6) and β (2→1)).

In particular, β-(2→6) fructooligosaccharides (FOSs) and fructooligomers exhibit higher prebiotic activity compared to commercial β-(2→1)-FOSs, owing to increased colonic persistence and selective fermentation. Accordingly, over the last few years research in the field of fructans revolved around production methods and scaling. However, further research in the therapeutic aspect of fructans is desired to elucidate further treatment methods with fructans.

The prevalence of obesity and hypertension has increased in the western world. Obesity (e.g. morbid obesity) can lead to diabetes, heart problems, hypertension, mental health issues, and many other associated complications. Hypertension can be detrimental to one's health and go as far as impair vision, lead to water accumulation in the lungs and cause erectile dysfunction. Often, individuals affected by hypertension are unaware of their condition. It would be advantageous to leverage the improvements in fructan production method and find new therapeutic uses such as the treatment of obesity and/or hypertension.

### SUMMARY

It was surprisingly found that levan, preferably low molecular weight levan, provides benefits (e.g. prevent, treat, or alleviate the symptoms) in addressing obesity and/or hypertension. Accordingly, in one aspect, there is provided a method of treating obesity and/or hypertension in a subject in need thereof comprising administering to the subject a therapeutically effective dose of levan. In a further aspect, there is provided a method of treating obesity and/or hypertension in a subject in need thereof comprising administering to the subject a pharmaceutical composition comprising levan and a pharmaceutically acceptable excipient. In still a further aspect, there is provided a levan for use in the manufacture of a medicament for the treatment of obesity and/or hypertension. In yet a further aspect, there is provided a pharmaceutical composition comprising levan and a pharmaceutically acceptable carrier for use in the treatment of obesity and/or hypertension, or for use in the manufacture of a medicament for the treatment of obesity and/or hypertension. In an additional aspect, there is provided a use of a levan in the treatment of obesity and/or hypertension or in the manufacture of a medicament for the treatment of obesity and/or hypertension. In still an additional aspect, there is provided a use of a pharmaceutical composition comprising a levan and a pharmaceutically acceptable excipient in the treatment of obesity and/or hypertension, or in the manufacture of a medicament for the treatment of obesity and/or hypertension. In some embodiments, the levan has a molecular weight of less than 20 kDa. In further embodiments, at least 85% of glycosidic linkages in the levan are β-[2,6]-glycosidic linkages.

Many further features and combinations thereof concerning the present improvements will appear to those skilled in the art following a reading of the instant disclosure.

### DESCRIPTION OF THE DRAWINGS

FIG. 1A is a combined ¹H and ¹³C nuclear magnetic resonance (NMR) spectrum for synthesized levan.

FIG. 1B is a ¹³C nuclear magnetic resonance (NMR) spectrum for synthesized levan.

FIG. 2A is a graph showing the body weight (BW) in function of time for control mice (1) having receiving a normal diet, experimental mice (2) having received a high fat diet (HFD) and low molecular weight levan, and control mice on HFD (3) that did not receive low molecular weight levan.

FIG. 2B is a bar graph showing the percent fat mass of control mice (C) with a normal diet, HFD mice, and experimental mice (EXP) having received a HFD and levan treatment.

FIG. 2C is a bar graph showing the percent lean mass of control mice (C) with a normal diet, HFD mice, and experimental mice (EXP) having received a HFD and levan treatment.

FIG. 2D is a bar graph showing the blood glucose (BG) of control mice (C) with a normal diet, HFD mice, and experimental mice (EXP) having received a HFD and levan treatment.

FIG. 2E is a bar graph showing the total cholesterol of control mice (C) with a normal diet, HFD mice, and experimental mice (EXP) having received a HFD and levan treatment.

FIG. 2F is a bar graph showing the low density lipoprotein (LDL) of control mice (C) with a normal diet, HFD mice, and experimental mice (EXP) having received a HFD and levan treatment.

FIG. 2G is a bar graph showing the high density lipoprotein (HDL) of control mice (C) with a normal diet, HFD mice, and experimental mice (EXP) having received a HFD and levan treatment.

FIG. 2H is a bar graph showing the triglyceride (TG) of control mice (C) with a normal diet, HFD mice, and experimental mice (EXP) having received a HFD and levan treatment.

FIG. 3A a graph showing the original traces for the harvesting of the thoracic aorta from mice C, HFD and EXP, and exposing the aortas to acetylcholine (Ach) in vitro using the wire myograph (cumulative doses of Ach after precontraction to phenylephrine).

FIG. 3B is a graph of the aorta relaxation percentage in function of the logarithm of the concentration of acetylcholine (endothelium dependent relaxation to Ach) for control mice (1), EXP mice (2) and HFD mice (3).

FIG. 3C is a graph of the aorta relaxation percentage in function of the logarithm of the concentration of sodium nitroprusside (SNP) (endothelium independent relaxation to SNP) for control mice (1), EXP mice (2) and HFD mice (3).

FIG. 3D is a western blot of phospho-endothelial nitric oxide synthase (P-eNOS), phosphorylated endothelial nitric oxide synthase (T-eNOS), binding protein (BIP), C/EBP homologous protein (CHOP), tumor necrosis factor alpha (TNFα), P65 and glyceraldehyde-3-phosphate dehydrogenase (GAPDH), for the control mice, the HFD mice and the EXP mice.

FIG. 3E is a bar graph showing the quantification of P-eNOS/T-eNOS based on the western blot of FIG. 3D, for the control mice, the HFD mice and the EXP mice.

FIG. 3F is a bar graph showing the quantification of P-eNOS/GAPDH based on the western blot of FIG. 3D, for the control mice, the HFD mice and the EXP mice.

FIG. 3G is a bar graph showing the quantification of P65/GAPDH based on the western blot of FIG. 3D, for the control mice, the HFD mice and the EXP mice.

FIG. 3H is a bar graph showing the quantification of TNFα/GAPDH based on the western blot of FIG. 3D, for the control mice, the HFD mice and the EXP mice.

FIG. 3I is a bar graph showing the quantification of BIP/GAPDH based on the western blot of FIG. 3D, for the control mice, the HFD mice and the EXP mice.

FIG. 3J is a bar graph showing the quantification of CHOP/GAPDH in the thoracic aorta based on the western blot of FIG. 3D, for the control mice, the HFD mice and the EXP mice.

FIG. 3K is a graph showing the mRNA level of BIP/GAPDH in the thoracic aorta for the control mice, HFD mice and EXP mice.

FIG. 3L is a graph showing the mRNA level of CHOP/GAPDH in the thoracic aorta for the control mice, HFD mice and EXP mice.

FIG. 3M is a graph showing the mRNA level of ATF4/GAPDH in the thoracic aorta for the control mice, HFD mice and EXP mice.

FIG. 3N is a graph showing the mRNA level of ATF6/GAPDH in the thoracic aorta for the control mice, HFD mice and EXP mice.

FIG. 3O is a graph showing the mRNA level of P65/GAPDH in the thoracic aorta for the control mice, HFD mice and EXP mice.

FIG. 3P is a graph showing the mRNA level of P50/GAPDH in the thoracic aorta for the control mice, HFD mice and EXP mice.

FIG. 3Q is a graph showing the mRNA level of TNFα/GAPDH in the thoracic aorta for the control mice, HFD mice and EXP mice.

FIG. 3R is a graph showing the mRNA level of VCAM1/GAPDH in the thoracic aorta for the control mice, HFD mice and EXP mice.

FIG. 3S is a graph showing the mRNA level of ICAM1/GAPDH in the thoracic aorta for the control mice, HFD mice and EXP mice.

FIG. 3T is a graph showing the mRNA level of NOX1/GAPDH in the thoracic aorta for the control mice, HFD mice and EXP mice.

FIG. 3U is a graph showing the mRNA level of NOX2/GAPDH in the thoracic aorta for the control mice, HFD mice and EXP mice.

FIG. 3V is a graph showing the mRNA level of NOX4/GAPDH in the thoracic aorta for the control mice, HFD mice and EXP mice.

FIG. 3W is a heat map showing all the markers in thoracic aorta from control mice and mice fed with HFD in the presence and absence of low levan β-[2,6]-glycosidic linkages treatment.

FIG. 4A shows a graph of the original traces for the harvesting of the mesenteric arteries from mice C, HFD and EXP, and exposing the mesenteric to Ach in vitro using the wire myograph (cumulative doses of Ach after precontraction to phenylephrine).

FIG. 4B is a graph of the mesenteric relaxation percentage in function of the logarithm of the concentration of acetylcholine (endothelium dependent relaxation to Ach) for control mice (1), EXP mice (2) and HFD mice (3).

FIG. 4C is a graph of the mesenteric relaxation percentage in function of the logarithm of the concentration of sodium nitroprusside (SNP) (endothelium independent relaxation to SNP) for control mice (1), EXP mice (2) and HFD mice (3).

FIG. 4D is a western blot of P-eNOS, T-eNOS, BIP, CHOP, TNFα, P65 and GAPDH, in mesenteric arteries for the control mice, the HFD mice and the EXP mice.

FIG. 4E is a bar graph showing the quantification of P-eNOS/T-eNOS in mesenteric arteries based on the western blot of FIG. 4D, for the control mice, the HFD mice and the EXP mice.

FIG. 4F is a bar graph showing the quantification of P-eNOS/GAPDH in mesenteric arteries based on the western blot of FIG. 4D, for the control mice, the HFD mice and the EXP mice.

FIG. 4G is a bar graph showing the quantification of P65/GAPDH in mesenteric arteries based on the western blot of FIG. 4D, for the control mice, the HFD mice and the EXP mice.

FIG. 4H is a bar graph showing the quantification of TNFα/GAPDH in mesenteric arteries based on the western blot of FIG. 4D, for the control mice, the HFD mice and the EXP mice.

FIG. 4I is a bar graph showing the quantification of BIP/GAPDH in mesenteric arteries based on the western blot of FIG. 4D, for the control mice, the HFD mice and the EXP mice.

FIG. 4J is a bar graph showing the quantification of CHOP/GAPDH in mesenteric arteries based on the western blot of FIG. 4D, for the control mice, the HFD mice and the EXP mice.

FIG. 5A is a graph showing the mRNA level of BIP/18S in proximal colon for the control mice, HFD mice and EXP mice.

FIG. 5B is a graph showing the mRNA level of CHOP/18S in proximal colon for the control mice, HFD mice and EXP mice.

FIG. 5C is a graph showing the mRNA level of ATF4/18S in proximal colon for the control mice, HFD mice and EXP mice.

FIG. 5D is a graph showing the mRNA level of p65/18S in proximal colon for the control mice, HFD mice and EXP mice.

FIG. 5E is a graph showing the mRNA level of P50/18S in proximal colon for the control mice, HFD mice and EXP mice.

FIG. 5F is a graph showing the mRNA level of TNFα/18S in proximal colon for the control mice, HFD mice and EXP mice.

FIG. 5G is a graph showing the mRNA level of VCAM/18S in proximal colon for the control mice, HFD mice and EXP mice.

FIG. 5H is a graph showing the mRNA level of ICAM/18S in proximal colon for the control mice, HFD mice and EXP mice.

FIG. 5I is a graph showing the mRNA level of NOX1/18S in proximal colon for the control mice, HFD mice and EXP mice.

FIG. 5J is a graph showing the mRNA level of NOX2/18S in proximal colon for the control mice, HFD mice and EXP mice.

FIG. 5K is a graph showing the mRNA level of NOX4/18S in proximal colon for the control mice, HFD mice and EXP mice.

FIG. 5L is a heat map showing all the markers in proximal colon from control mice and mice fed with HFD in the presence and absence of low levan β-[2,6]-glycosidic linkages treatment.

FIG. 6A is a graph showing the mRNA level of BIP/18S in distal colon for the control mice, HFD mice and EXP mice.

FIG. 6B is a graph showing the mRNA level of CHOP/18S in distal colon for the control mice, HFD mice and EXP mice.

FIG. 6C is a graph showing the mRNA level of ATF4/18S in distal colon for the control mice, HFD mice and EXP mice.

FIG. 6D is a graph showing the mRNA level of p65/18S in distal colon for the control mice, HFD mice and EXP mice.

FIG. 6E is a graph showing the mRNA level of P50/18S in distal colon for the control mice, HFD mice and EXP mice.

FIG. 6F is a graph showing the mRNA level of TNFα/18S in distal colon for the control mice, HFD mice and EXP mice.

FIG. 6G is a graph showing the mRNA level of VCAM/18S in distal colon for the control mice, HFD mice and EXP mice.

FIG. 6H is a graph showing the mRNA level of ICAM/18S in distal colon for the control mice, HFD mice and EXP mice.

FIG. 6I is a graph showing the mRNA level of NOX1/18S in distal colon for the control mice, HFD mice and EXP mice.

FIG. 6J is a graph showing the mRNA level of NOX2/18S in distal colon for the control mice, HFD mice and EXP mice.

FIG. 6K is a graph showing the mRNA level of NOX4/18S in distal colon for the control mice, HFD mice and EXP mice.

FIG. 6L is a heat map showing all the markers in distal colon from control mice and mice fed with HFD in the presence and absence of low levan β-[2,6]-glycosidic linkages treatment.

FIG. 7A is a graph showing the level of Alanine Aminotransferase/SGPT (ALT) in control mice, HFD mice and EXP mice.

FIG. 7B is a graph showing the level of alkaline phosphatase (ALP) in control mice, HFD mice and EXP mice.

FIG. 8A is a graph showing the systolic blood pressure (BP) in function of time for control mice administered vehicle (1), EXP mice administered angiotensin II and levan (2) and mice administered angiotensin II without levan (3).

FIG. 8B is a graph showing the body weight in function of time for control mice administered vehicle (●), EXP mice administered angiotensin II and levan (▴) and mice administered angiotensin II without levan (▪).

FIG. 8C is a graph showing the aorta contraction percentage in response to the log of phenylephrine (PE) concentration for control mice administered vehicle (1), EXP mice administered angiotensin II and levan (2) and mice administered angiotensin II without levan (3).

FIG. 8D is a graph showing the aorta contraction percentage in response to the log of thromboxane A analog (TXA) concentration for control mice administered vehicle (1), EXP mice administered angiotensin II and levan (2) and mice administered angiotensin II without levan (3).

FIG. 8E is a graph showing the aorta contraction percentage in response to the log of angiotensin II (Ang II) concentration for control mice administered vehicle (1), EXP mice administered angiotensin II and levan (2) and mice administered angiotensin II without levan (3).

FIG. 8F is a graph showing the aorta relaxation percentage in function of the logarithm of the concentration of acetylcholine (endothelium dependent relaxation to Ach) for control mice administered vehicle (1), EXP mice administered angiotensin II and levan (2) and mice administered angiotensin II without levan (3).

FIG. 8G is a graph of the aorta relaxation percentage in function of the logarithm of the concentration of sodium nitroprusside (SNP) (endothelium independent relaxation to SNP) for control mice administered vehicle (1), EXP mice administered angiotensin II and levan (2) and mice administered angiotensin II without levan (3).

FIG. 8H is a western blot of IP₃R1,p-Protein kinase-like endoplasmic reticulum kinase (PERK), t-PERK, P-eNOS, T-eNOS, BIP, NOX1, TNFα, P65, cytochrome c and GAPDH, in thoracic aorta for control mice administered vehicle (CTL), EXP mice administered angiotensin II and levan (A+L), and mice administered angiotensin II without levan (ANG).

FIG. 8I is a bar graph showing the quantification of IP₃R1/GAPDH based on the western blot of FIG. 8H, for the control mice administered vehicle, EXP mice administered angiotensin II and levan, and mice administered angiotensin II without levan.

FIG. 8J is a bar graph showing the quantification of p-PERK/t-PERK based on the western blot of FIG. 8H, for the control mice administered vehicle, EXP mice administered angiotensin II and levan, and mice administered angiotensin II without levan.

FIG. 8K is a bar graph showing the quantification of p-eNOS/t-eNOS based on the western blot of FIG. 8H, for the control mice administered vehicle, EXP mice administered angiotensin II and levan, and mice administered angiotensin II without levan.

FIG. 8L is a bar graph showing the quantification of BIP/GAPDH based on the western blot of FIG. 8H, for the control mice administered vehicle, EXP mice administered angiotensin II and levan, and mice administered angiotensin II without levan.

FIG. 8M is a bar graph showing the quantification of NOX1/GAPDH based on the western blot of FIG. 8H, for the control mice administered vehicle, EXP mice administered angiotensin II and levan, and mice administered angiotensin II without levan.

FIG. 8N is a bar graph showing the quantification of TNFα/GAPDH based on the western blot of FIG. 8H, for the control mice administered vehicle, EXP mice administered angiotensin II and levan, and mice administered angiotensin II without levan.

FIG. 8O is a bar graph showing the quantification of cytochrome c/GAPDH based on the western blot of FIG. 8H, for the control mice administered vehicle, EXP mice administered angiotensin II and levan, and mice administered angiotensin II without levan.

FIG. 8P is a bar graph showing the quantification of p65/GAPDH based on the western blot of FIG. 8H, for the control mice administered vehicle, EXP mice administered angiotensin II and levan, and mice administered angiotensin II without levan.

FIG. 8Q is a bar graph showing the miR-204/RNU6 expression, in thoracic aorta from control mice administered vehicle, EXP mice administered angiotensin II and levan, and mice administered angiotensin II without levan.

FIG. 8R is a graph showing the mRNA level of IP₃R1/GAPDH in thoracic aorta from control mice administered vehicle, EXP mice administered angiotensin II and levan, and mice administered angiotensin II without levan.

FIG. 8S is a graph showing the mRNA level of NOX1/GAPDH in thoracic aorta from control mice administered vehicle, EXP mice administered angiotensin II and levan, and mice administered angiotensin II without levan.

FIG. 8T is a graph showing the mRNA level of NOX2/GAPDH in thoracic aorta from control mice administered vehicle, EXP mice administered angiotensin II and levan, and mice administered angiotensin II without levan.

FIG. 8U is a graph showing the mRNA level of NOX4/GAPDH in thoracic aorta from control mice administered vehicle, EXP mice administered angiotensin II and levan, and mice administered angiotensin II without levan.

FIG. 8V is a graph showing the mRNA level of TNFα/GAPDH in thoracic aorta from control mice administered vehicle, EXP mice administered angiotensin II and levan, and mice administered angiotensin II without levan.

FIG. 8W is a graph showing the mRNA level of VCAM/GAPDH in thoracic aorta from control mice administered vehicle, EXP mice administered angiotensin II and levan, and mice administered angiotensin II without levan.

FIG. 8X is a graph showing the mRNA level of ICAM/GAPDH in thoracic aorta from control mice administered vehicle, EXP mice administered angiotensin II and levan, and mice administered angiotensin II without levan.

FIG. 8Y is a graph showing the mRNA level of ATF4/GAPDH in thoracic aorta from control mice administered vehicle, EXP mice administered angiotensin II and levan, and mice administered angiotensin II without levan.

FIG. 8Z is a graph showing the mRNA level of ATF6/GAPDH in thoracic aorta from control mice administered vehicle, EXP mice administered angiotensin II and levan, and mice administered angiotensin II without levan.

FIG. 9A is a graph showing the mRNA level of BIP/18S for the proximal colon from control mice administered vehicle, EXP mice administered angiotensin II and levan, and mice administered angiotensin II without levan.

FIG. 9B is a graph showing the mRNA level of CHOP/18S for the proximal colon from control mice administered vehicle, EXP mice administered angiotensin II and levan, and mice administered angiotensin II without levan.

FIG. 9C is a graph showing the mRNA level of ATF4/18S for the proximal colon from control mice administered vehicle, EXP mice administered angiotensin II and levan, and mice administered angiotensin II without levan.

FIG. 9D is a graph showing the mRNA level of p65/18S for the proximal colon from control mice administered vehicle, EXP mice administered angiotensin II and levan, and mice administered angiotensin II without levan.

FIG. 9E is a graph showing the mRNA level of P50/18S for the proximal colon from control mice administered vehicle, EXP mice administered angiotensin II and levan, and mice administered angiotensin II without levan.

FIG. 9F is a graph showing the mRNA level of TNFα/18S for the proximal colon from control mice administered vehicle, EXP mice administered angiotensin II and levan, and mice administered angiotensin II without levan.

FIG. 9G is a graph showing the mRNA level of VCAM/18S for the proximal colon from control mice administered vehicle, EXP mice administered angiotensin II and levan, and mice administered angiotensin II without levan.

FIG. 9H is a graph showing the mRNA level of NOX1/18S for the proximal colon from control mice administered vehicle, EXP mice administered angiotensin II and levan, and mice administered angiotensin II without levan.

FIG. 9I is a graph showing the mRNA level of NOX2/18S for the proximal colon from control mice administered vehicle, EXP mice administered angiotensin II and levan, and mice administered angiotensin II without levan.

FIG. 9J is a graph showing the mRNA level of NOX4/18S for the proximal colon from control mice administered vehicle, EXP mice administered angiotensin II and levan, and mice administered angiotensin II without levan.

FIG. 9K is a heat map showing the markers in proximal colon from normotensive mice and hypertensive mice treated with and without low levan β-[2,6]-glycosidic linkages treatment.

FIG. 10A is a graph showing the mRNA level of BIP/18S for the distal colon from control mice administered vehicle, EXP mice administered angiotensin II and levan, and mice administered angiotensin II without levan.

FIG. 10B is a graph showing the mRNA level of CHOP/18S for the distal colon from control mice administered vehicle, EXP mice administered angiotensin II and levan, and mice administered angiotensin II without levan.

FIG. 10C is a graph showing the mRNA level of ATF4/18S for the distal colon from control mice administered vehicle, EXP mice administered angiotensin II and levan, and mice administered angiotensin II without levan.

FIG. 10D is a graph showing the mRNA level of p65/18S for the distal colon from control mice administered vehicle, EXP mice administered angiotensin II and levan, and mice administered angiotensin II without levan.

FIG. 10E is a graph showing the mRNA level of P50/18S for the distal colon from control mice administered vehicle, EXP mice administered angiotensin II and levan, and mice administered angiotensin II without levan.

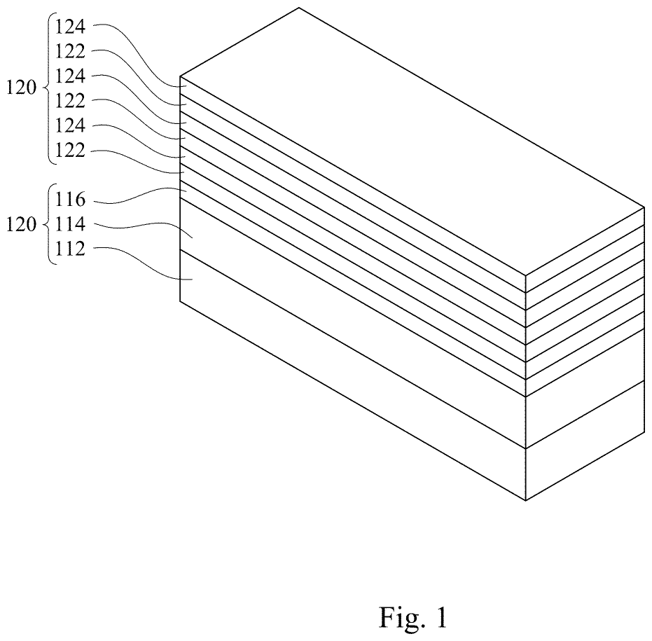

FIG. 1OF is a graph showing the mRNA level of TNFα/18S for the distal colon from control mice administered vehicle, EXP mice administered angiotensin II and levan, and mice administered angiotensin II without levan.

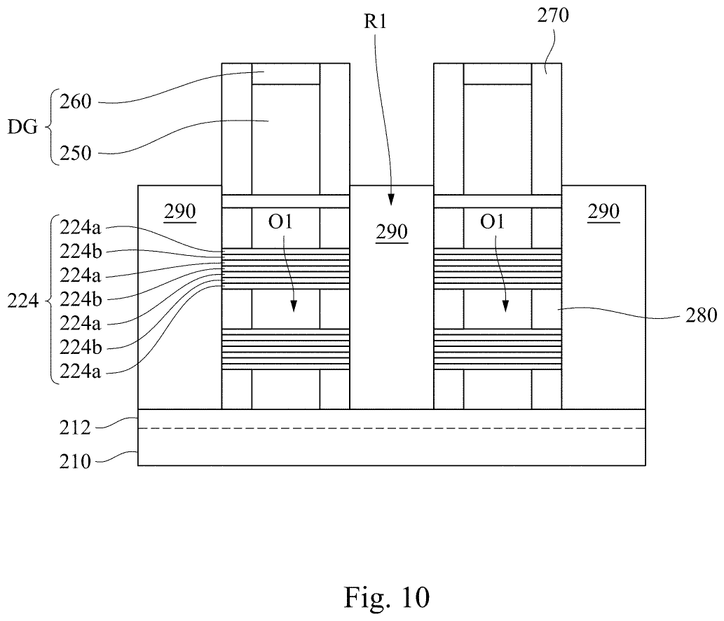

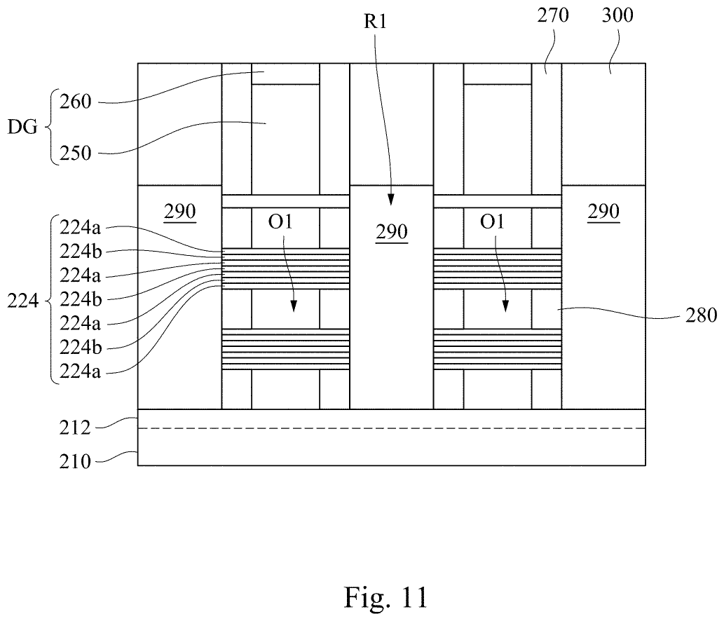

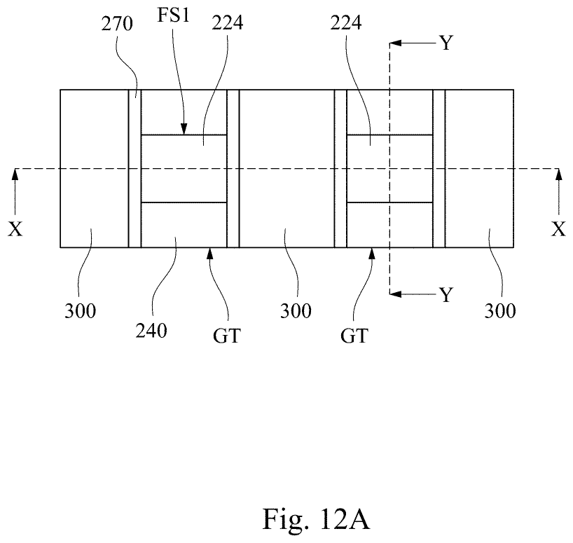

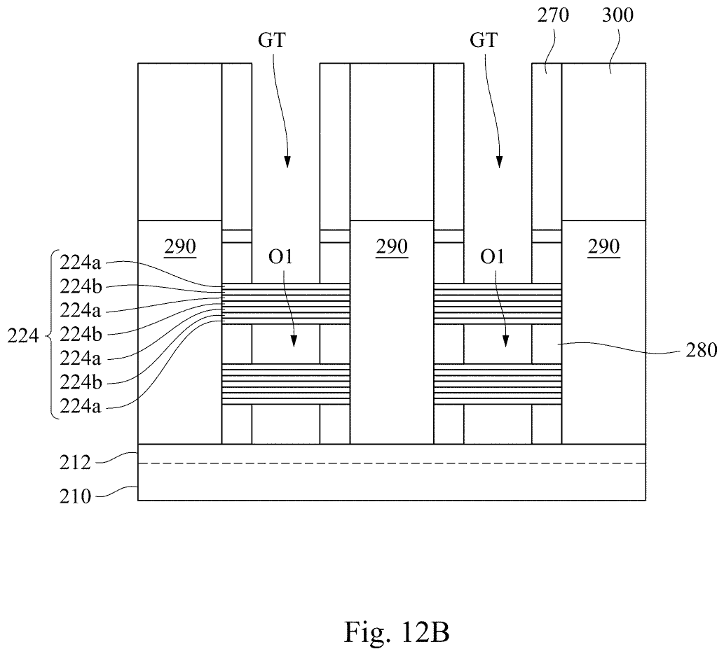

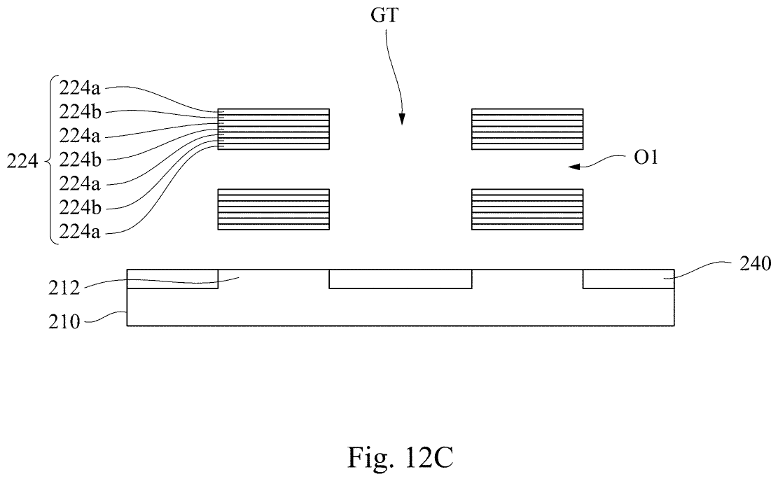

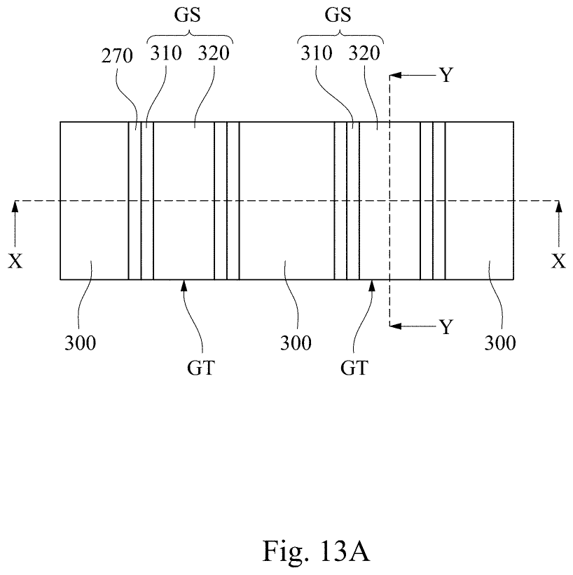

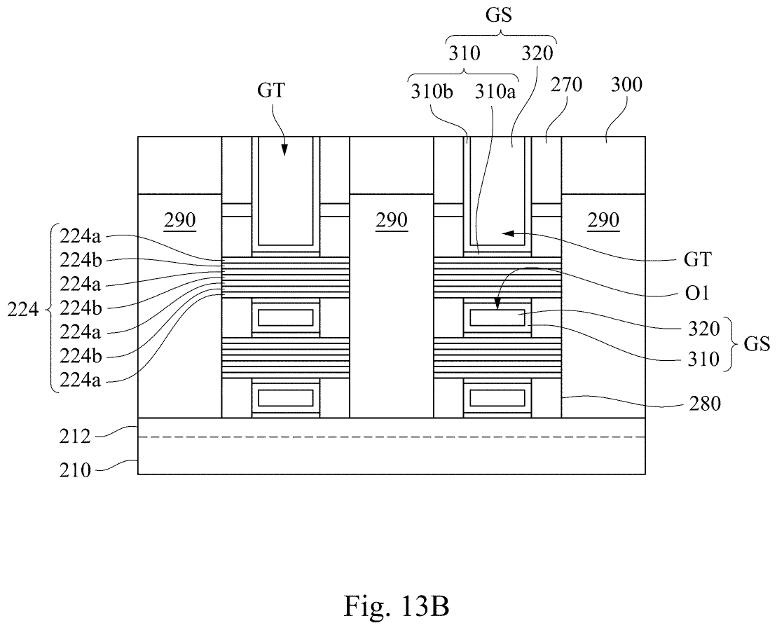

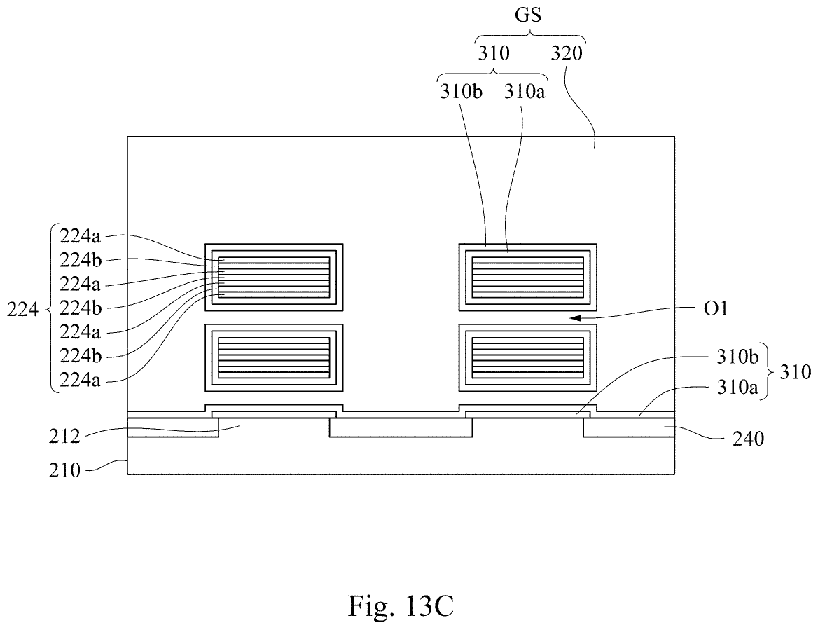

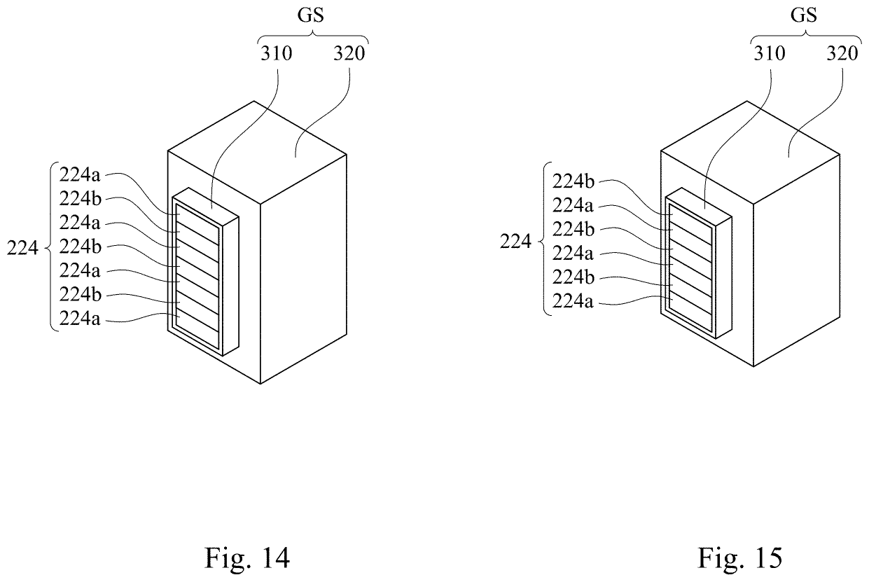

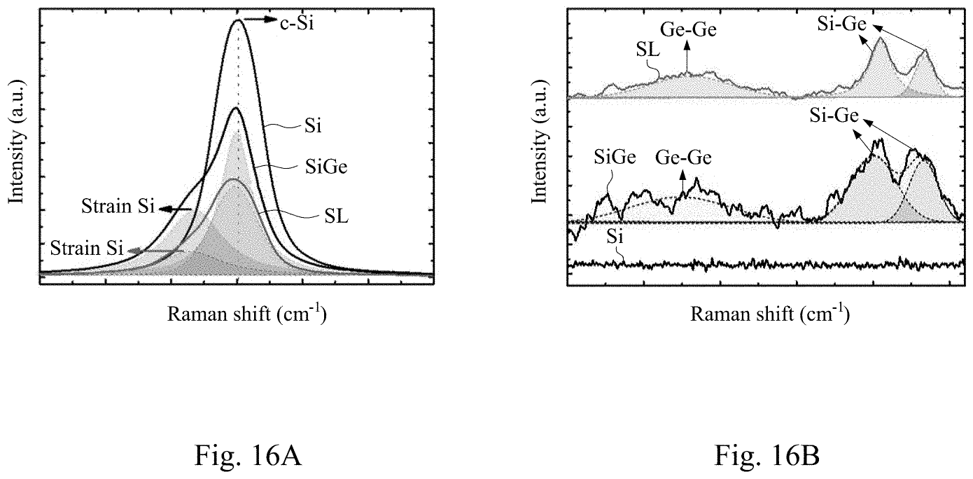

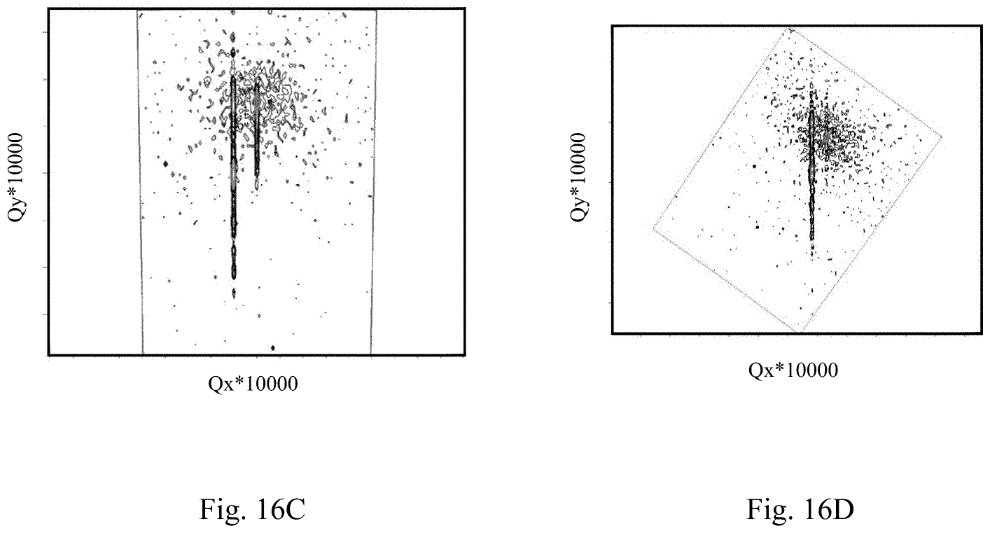

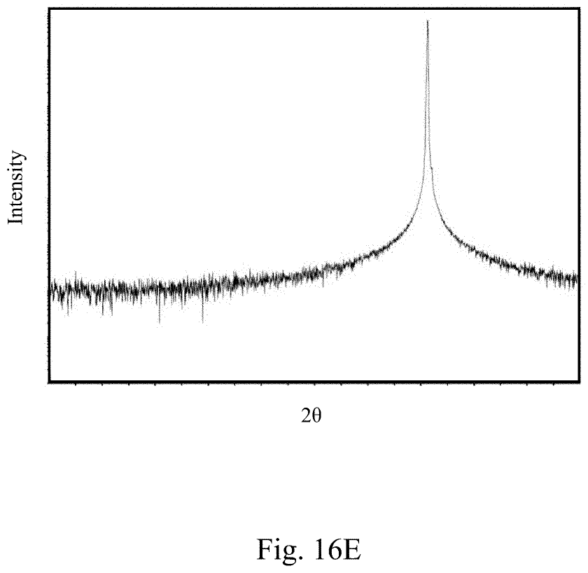

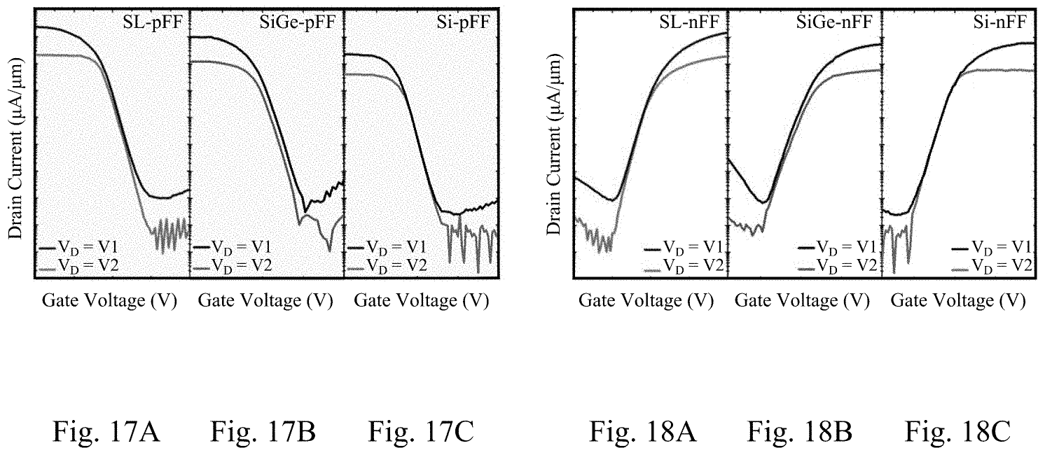

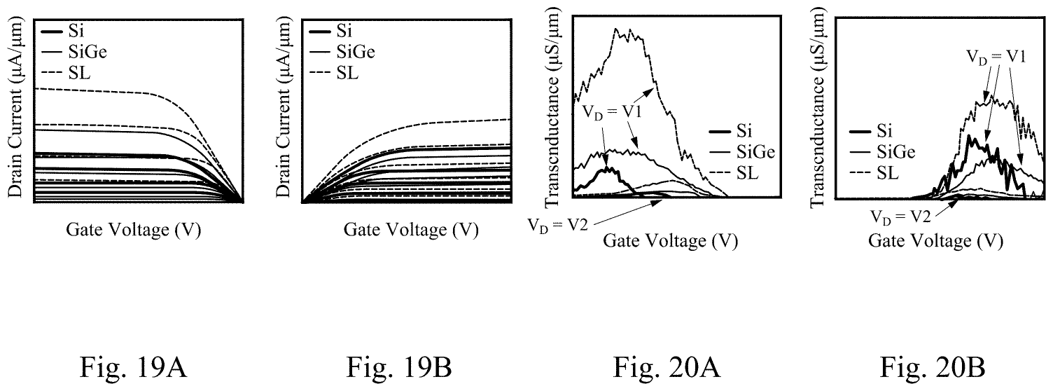

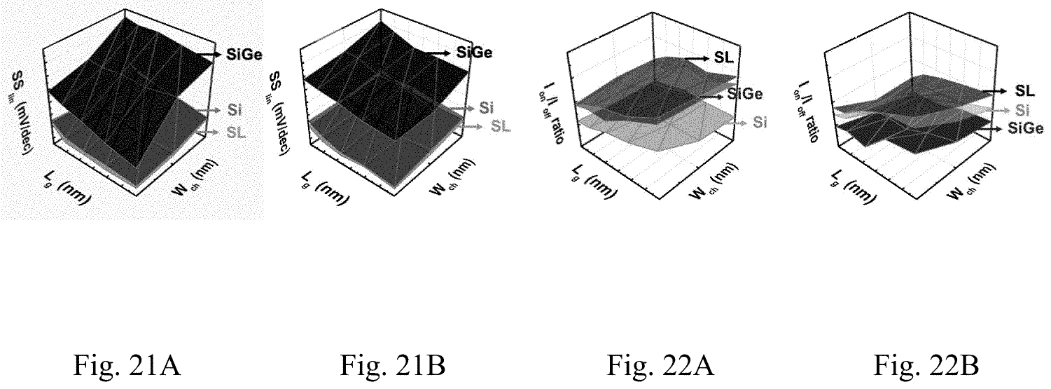

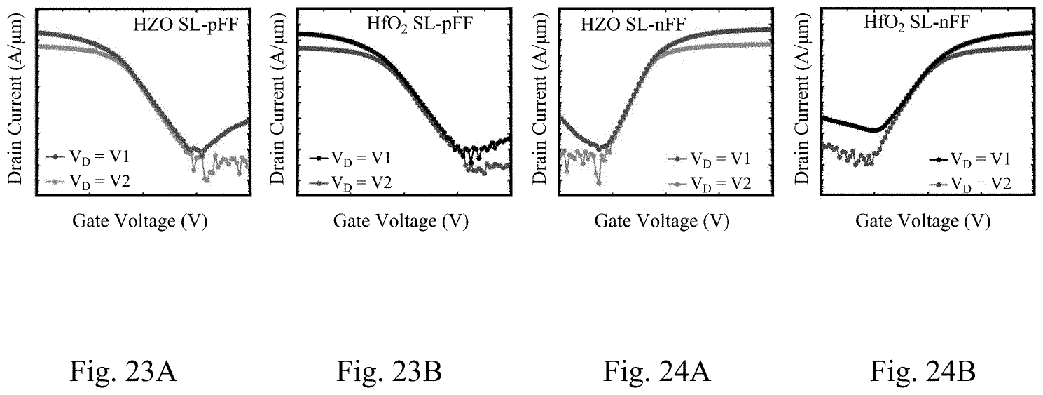

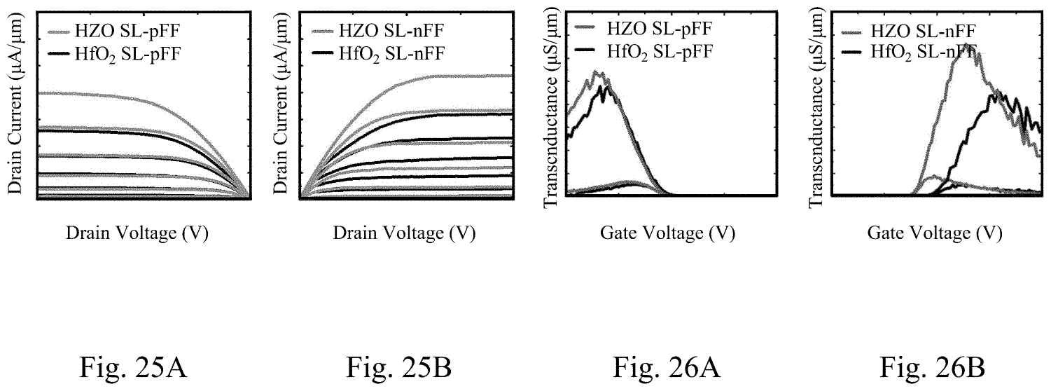

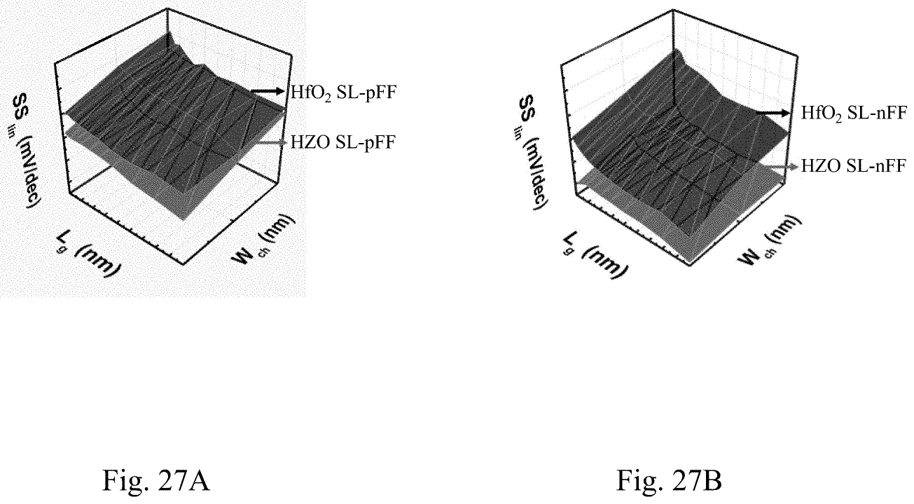

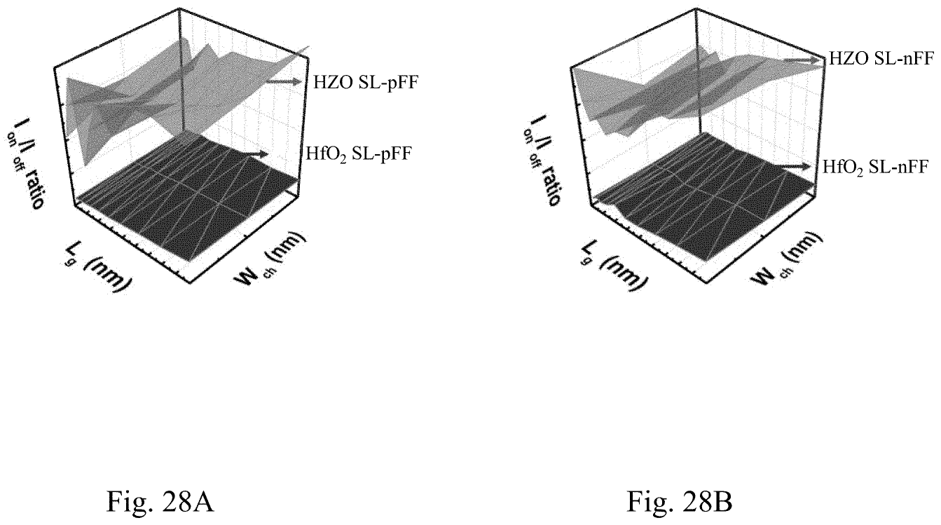

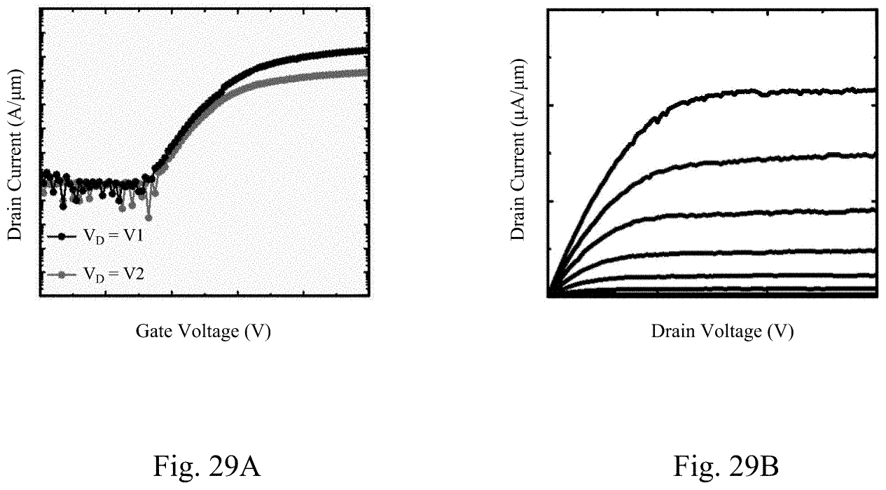

FIG. 10G is a graph showing the mRNA level of VCAM/18S for the distal colon from control mice administered vehicle, EXP mice administered angiotensin II and levan, and mice administered angiotensin II without levan.

FIG. 10H is a graph showing the mRNA level of NOX1/18S for the distal colon from control mice administered vehicle, EXP mice administered angiotensin II and levan, and mice administered angiotensin II without levan.

FIG. 10I is a graph showing the mRNA level of NOX2/18S for the distal colon from control mice administered vehicle, EXP mice administered angiotensin II and levan, and mice administered angiotensin II without levan.

FIG. 10J is a graph showing the mRNA level of NOX4/18S for the distal colon from control mice administered vehicle, EXP mice administered angiotensin II and levan, and mice administered angiotensin II without levan.

FIG. 10K is a heat map showing the markers in distal colon from normotensive mice and hypertensive mice treated with and without low levan β-[2,6]-glycosidic linkages treatment.

FIG. 11A is a graph showing the level of Alanine Aminotransferase/SGPT (ALT) in control mice administered vehicle, EXP mice administered angiotensin II and levan, and mice administered angiotensin II without levan.

FIG. 11B is a graph showing the level of alkaline phosphatase (ALP) in control mice administered vehicle, EXP mice administered angiotensin II and levan, and mice administered angiotensin II without levan.

### DETAILED DESCRIPTION

The present disclosure provides methods of using levans to treat obesity and/or hypertension in a subject in need thereof. Levans are prebiotics which are non-digestible carbohydrates. These carbohydrates are able to bypass the upper gastrointestinal tract, by resisting the hydrolysis of digestive enzymes and absorption, and reach the colon where they are fermented by the gut microflora. The levans can be obtained using one of three methods: plant extraction, chemical synthesis, or enzymatic production.

The term “levan” as used herein is to be understood as fructans having β (2→6) glycosidic linkages. The term levan as used herein can include fructans having glycosidic linkages that consist essentially of β (2→6) linkages. The term levan as used herein can include fructooligosaccharides (FOSs) having the majority of their glycosidic linkages being β (2→6) glycosidic linkages compared to β-(2→1) linkages (e.g. at least 50%, at least 60%, at least 70%, at least 80%, at least 85% or at least 90%). In preferred embodiments, the levan according to the present disclosure has at least 85% of its glycosidic linkages being β (2→6) glycosidic linkages compared to β-(2→1) linkages. In some embodiments, the levan is a low molecular weight levan having a ratio of terminal units of fructose to glucose being in the range of from 4 to 5.

The levan of the present disclosure is preferably a low molecular weight levan. The low molecular weight can be defined as a molecular weight in the range of less than 2×10⁴ Da, less than 10⁴ Da, less than 5×10³, less than 10³. In some embodiments, the molecular weight of the low molecular weight levan is comprised in the range of from 5 to 20 kDa.

Chemical synthesis can be used to produce FOSs in two ways: polymerization of monosaccharides and hydrolyzation of polysaccharides. In a bottom-up strategy in which the synthesis begins from the monomers, considering they have various functional groups and chiral centers, selective protection-deprotection steps are necessary to control the stereochemical and regiochemical specificity of the desired glycosidic bonds. Moreover, the chemical synthesis of FOSs is a multi-step process with laborious and costly procedures and involves toxic reagents that are not safe to use based on food safety guidelines. In a top-down strategy in which the synthesis begins from polysaccharides, selective chemical hydrolysis is challenging to achieve, accordingly a complex mixture of products may be produced containing brown contaminants resulting from the conventional heating procedure.

There are two strategies for the enzymatic synthesis of levans. The first one is the bottom-up strategy. The enzymes are used to produce levans by transfructosylation from simple saccharides containing fructose such as sucrose to produce FOSs and corresponding polymers. β-fructofuranosidases (EC 3.2.1.26) and fructosyl-transferases (EC 2.4.1) are two groups of enzymes that follow the bottom-up strategy by cleaving fructosyl moieties from simple carbohydrates and coupling them to obtain a higher degree of polymerization. The second strategy is top-down in which high molecular levans undergo controlled hydrolyzation by the use of fructanases. A high molecular weight levan may be defined as having a molecular weight of at least 60 kDa.

In some embodiments, a bi-enzymatic process (end-levanase & Levansucrase) can be used for the production of beta-2-6-oligolevans with a controlled size. The bi-enzymatic system was developed from combining immobilized levanase from Capnocytophaga ochracea (LEV) and the immobilized levansucrase from Bacillus amyloliquefaciens (LS-B.A.) on Gly-Ag-IDA/Cu, which exhibit superior ability in levan synthesis. The potential interference of the selected enzymes was investigated, and the results showed an interference between the LS and LEV towards sucrose. Based on the interference, the ratios of the LS and LEV were adjusted in the bi-enzymatic systems. The two-step, one-step, and co-immobilized bi-enzymatic systems were assessed for FOSs synthesis from sucrose. The two-step bi-enzymatic reaction (1:1 LS/LEV) resulted in the highest oligo yield and enzyme selectivity by producing 45.7% (w/w) of GF7 after 48 h incubation at 15 oC, while the co-immobilized system (with the initial ratio of 1 U:0.67 U LS/LEV) ended up the lowest yield and highest relative proportion of GF2 (10.8% and 23.2% respectively). The two-step bi-enzymatic system produced a detectable level of levans during the bi-enzymatic reaction which indicated the importance of the primary incubation time for levan formation by the LS to achieve higher oligo yields. Moreover, the product profile study showed the synthesis of levan by LS continued to happen even after the addition of LEV to reach a maximum yield of 24.8% (w/w) after 5 h followed by a decrease in the levan yield and a significant increase in the oligo yield (45.7%, w/w) at 48 h. The use of immobilized LS-B.A. favored the synthesis of HMW levans (>10000 kDa) by producing the highest ratio of the levans at the beginning of the bi-enzymatic reaction. The two-step immobilized bi-enzymatic system was used for conducting a response surface morphology (RSM) optimization by applying a five-level, two variable central composite rotatable design (CORD). According to the statistical calculation, applying 15 h and 50% proportion would result in the maximum oligo yield (63%, w/w) indicating the credibility of the effects of the incubation time and the LS proportion. Indeed, the most important factor in oligo yield improvement was the primary incubation time at which the LS carried out the levan synthesis. In the case of short first step incubation time, the presence of short-chain levans can promote excessive hydrolyzing activity of the levanase that decreased the oligo yield; also, long first step incubation time can result in oligo yield decrease but due to suppressed levanase activity.

Fructooligosaccharies are made up of 3 to 10 monosaccharides including fructose monomers with β (2→1) or β (2→6) glycosidic bond and often contain a terminal D-glucose joined by a α (1→2) glycosidic linkage. They are usually found in fruits and vegetables such as banana, onion, chicory root, garlic, asparagus, jicama, and leeks. Although some grains and cereals such as wheat and barley contain FOS, the Jerusalem artichoke and its relative yacon as well as the Blue Agave plant generally have the highest concentrations of FOS of cultured plants.

FOSs can be classified as inulin-type, neoinulin-type, levan-type, neolevan-type and mixed levan-type. In inulin-type, D-fructosyl units are attached by β (2→1) glycosidic linkages and the simplest molecular structure in this class is 1-kestose, a trisaccharide with terminal glucose joined with an α (1→2) glycosidic bond. In levan-type, the glycosidic bond is β (2→6) joining the fructosyl moieties and the simplest compound is 6-kestose, a trisaccharide with a terminal glucosyl moiety attached through an α (2→1) glycosidic bond. In either neoinulin- or neolevan-types, the core structure is a glucoside moiety attached to the fructosyl moieties through β (2→1) or β (2→6) glycosides linkages. The most basic compound in this category is neokestose. Finally, the mixed levan-type has both β (1→2) and β (2→6) glycosides linkages between fructosyl moieties, but the glucoside moiety is the terminal group and not the core structure. The basic structure in this class is a tetrasaccharide called bifurcose in which the core structure, fructose, is joined by two other fructoses via β (2→1) and β (2→6) glycosidic bonds respectively and a terminal glucoside moiety is attached by an α (2→6) glycosidic linkage. The method of treatment of the present disclosure only contemplates the use of FOS levan-type and mixed FOS levan-type from the subclasses of FOS. The FOS levan-type and FOS mixed levan-type should contain a majority of β (2→6).

The present disclosure provides methods of using levans in the treatment, prevention, or alleviation of symptoms for obesity and/or hypertension. Obesity represents a risk/predisposition for a subject to develop hypertension, but hypertension can be caused by many other factors. The factors that influence hypertension include but are not limited to a high salt diet, smoking, lack of physical activity, excessive alcohol consumption, old age, genetic predisposition. Obesity can be caused or influenced by factors including but not limited to a genetic predisposition, lack of physical activity, lack of control over quality and quantity of nutritional intake, lack of physical activity, high stress levels, certain drugs (such as steroid hormones), and mental health. In some embodiments, the present method can reduce the body weight of the subject treated, reduce fat mass and increase lean mass. The levans can have additional health benefit including but not limited to reducing inflammation, reducing oxidative stress, and endoplasmic reticulum (ER) stress in the cells and improving vascular function.

More generally, the present disclosure provides a method for the prevention, treatment and/or alleviation of hypertension and/or obesity comprising administering to a subject in need thereof a therapeutically effective amount of levans as defined herein. In some embodiments, additional treatment agents may be used in addition to levans for the treatment, prevention, and/or alleviation of symptoms of obesity and/or hypertension.

The administration of the levans can be an oral administration, but other administration routes are contemplated by the present disclosure. The levans described herein can be administered in any suitable manner, preferably with pharmaceutically acceptable carriers or excipients. The terms “pharmaceutically acceptable carrier”, “excipients”, “physiologically acceptable vehicle” and the like are to be understood as referring to an acceptable carrier that may be administered to a subject, together with the levan, and which does not destroy the pharmacological activity of levan. Further, as used herein “pharmaceutically acceptable carrier” or “pharmaceutical carrier” are known in the art and include, but are not limited to, a tablet (e.g. a chewable tablet, a swallow capsule, a dissolvable tablet), a powder, or a liquid phase (e.g. solution, suspension, emulsion etc.). Preservatives and other additives may also be present, such as, for example, antimicrobials, antioxidants, collating agents, inert gases and the like.

In some embodiments, there is provided a pharmaceutical composition comprising a levan as defined herein and a pharmaceutically acceptable carrier. The pharmaceutical composition can be used in the prevention, alleviation or treatment methods described herein. As used herein, “pharmaceutical composition” means therapeutically effective amounts (dose) of the compound together with pharmaceutically acceptable diluents, preservatives, solubilizers, emulsifiers, and/or carriers. A “therapeutically effective amount” as used herein in the context of the pharmaceutical composition refers to that amount which provides a therapeutic effect for a given condition and administration regimen.

The excipient(s) or carrier(s) must be “pharmaceutically acceptable” in the sense of being compatible with the other ingredients of the formulation and not being deleterious to the recipient thereof. Standard accepted excipient(s) or carrier(s) are well known to skilled practitioners and described in numerous textbooks.

It will be clear to a person skilled in the art that the amount of levan described herein and used in accordance with the disclosure can be determined by the attending physician or pharmacist. It will be appreciated that the amount of levan required will vary not only with the particular fructans subtype selected but also with the route of administration, the condition being treated, and the age of the subject. It will be understood that the scope of the method of treatment or uses described herein is not particularly limited, but includes in principle any therapeutically useful outcome including preventing, treating or slowing the progression of conditions defined herein such as obesity and hypertension. It will be understood that the levan can be administered as a dietary supplement in an off-the shelf type medicament.

### Example 1: Synthesis of Levan

Low molecular weight (LMW) levans were produced from sucrose using levansucrase (LS) monoenzymatic system or LS/Levanase bienzymatic system. For the monoenzymatic system, the enzymatic reactions were carried out in the presence of 0.9 U/ml of LS (from Bacillus amyloliquefaciens or from Gluconobacter oxydans) and 0.5 M sucrose in the phosphate buffered saline at pH of 6 and room temperature for 48 hours. For the bi-enzymatic system, free or immobilized LS from Bacillus amyloliquefaciens on Gly-Ag-IDA/Cu and free or immobilized levanase from Capnocytophaga ochracea on Gly-Ag-IDA at 0.6 U/ml:0.6 U/ml ratio were sequentially added to 0.6 M sucrose at pH of 6 and 15° C.; the first step was carried out for 15 h, while the second step was run for 48 hrs. To recover levans, ethanol was added to the reaction mixtures at a 2:1 (v/v) ratio, left overnight, and centrifuged at 9800 g for 20 min. The recovered levans were dialyzed against water through a membrane with a cut-off of 1000 Da at 4° C. Levans were then freeze-dried and stored at −80° C.

To determine MW distribution of polysaccharides, levans were characterized by high-pressure size-exclusion chromatography (HPSEC) using a Waters HPLC system equipped with a 1525 binary pump, refractometer 2489 detector, Breeze™ 2 software and TSK gel G5000PWXL-CP. The elution was carried out with 200 mM NaCl at a flow rate of 0.5 ml/min, using levan as a standard of MWs that range from 5 to 5124 kDa.

Nuclear magnetic resonance (NMR) spectroscopy was used to determine the glycosidic linkage type of levan produced. ¹H (800 MHz) and ¹³C (200 MHz) NMR spectra were recorded using an Avance III HD spectrometer (Bruker Corp., Billerica, MA, USA) equipped with a TCI cryoprobe. All two-dimensional heteronuclear spectra (HSQC and HSQC-TOCSY) were performed using standard pulse sequences available in the Bruker software. Three-dimensional HSQC-TCOSY data was collected using 25% non-uniform sampling. Chemical shifts were measured at 328 K in D₂O. All the experiments were performed by using 2,2-Dimethyl-2-silapentane-5-sulfonate as the internal standard. Chemical shifts were interpreted in the carbohydrate structure context by comparison with the standard in the literature.

To determine the linearity and branching ratios of levans' glycosidic linkages, methylation of levans was conducted and followed by gas chromatography (GC) analysis. A sample of levans (250 μg) was dissolved in dimethyl sulfoxide followed by treatment with NaOH powder. The levan sample was then fully dissolved by sonicating for 50 minutes, then methylated by adding CH₃l in an ice bath. The methylated levan was hydrolyzed with trifluoroacetic acid, then it was reduced with NaBD₄ and acetylated into partially methylated alditol acetates (PMAAs) as described by Anumula and Taylor (1992). Samples (1 μl) were injected in a splitless mode in a gas chromatograph (Agilent, Santa Clara, CA, USA) with Agilent DB-5HT column (30 m×250 μm×0.1 μm). Linkage type was determined by comparing the electron ionization—mass spectrometry (EI-MS) fragment pattern with the National Institute of Standards Technology library and the PMAAs database from the Complex Carbohydrate Research Center.

Table 1 shows the different types of levan produced upon LS catalyzing the transfructosylation reaction of sucrose. High molecular weight (HMW) levan (1700-5700 kDa) was produced by LS from G. oxydans-catalyzing the transfructosylation of sucrose at pH of 5, while mix L/HMW levan was produced using the same LS from G. oxydans at pH of 6. LMW levans were obtained upon the transfructosylation reaction of sucrose catalyzed by LS from B. amyloliquefaciens at a pH of 6 (Table 1). Using LSs from different bacterial sources and reaction conditions (pH) resulted in levans with different MW.

TABLE 1
Characteristics and properties of different levans,
as determined by NMR and methylation-GC analysis.

| | | | Structure | | |
|---|---|---|---|---|---|
| | MW | | Terminal | Linear | Branching |
| Levan | distri- | | unit ratio | unit ratio | unit ratio |
| Classi- | bution | Linkage | [2-Fru/ | [(2,6)- | [(1,2,6)- |
| fication* | (kDa) | types | 1-Glc] | Fru] | Fru] |
| High MW | 1700-5700 | β-2,6 | 1.34 | 11.6 | 1 |
| (HMW) | | | | | |
| Low MW | 3-8 | β-2,6 | 4.525 | 6.445 | 1 |
| extracellular | | | | | |
| Mix | 860-2700 | β-2,6 | 1.43 | 6.4 | 1 |
| (low-high) | (high) | | | | |
| | 4-5 (low) | | | | |

NMR analysis was carried out to characterize the type of glycosidic linkages of levans obtained from the transfructosylation reaction of sucrose catalyzed by LSs (FIGS. 1A-1B). The 1D-¹H NMR spectrum confirmed the absence of anomeric proton signals, indicating that the levan structures are made up of fructose units (FIG. 1A). The 1D ¹³C NMR spectrum of HMW, LMW and mix L/HWM levans (FIG. 1B) prove the structure of levan with [→6)-β-Fruc-(2→] linkages. The methylation and GC analysis of levans confirmed the presence of β-(2→6) fructosyl linkages (linear units) and 1,2,6-fructose linkages (branch units) and reducing end (2-fructose and 1-glucose units) in all levans (Table 1). The higher the MW of levans is, the higher the ratio of linear β-(2→6) fructosyl units to reducing ends and branching units is. Indeed, HMW levan is characterized by a ratio of 11.6:1.34:1 for linear, reducing ends to branching units, respectively. The results also confirmed the presence of β-(2,6) fructosyl linkages (linear units, 55%) and 1,2,6-fructose linkages (branch units, 8.5%) and reducing end (2-fructose and/or 1-glucose units, 36.5%) in LMW levans. The molecular weight distribution of LMW was about 3 to 8 kDa.

### Example 2: Treatment of Levan Using a Mouse Model

Eight-week-old C57/b6 male mice (stock number 000664, Jackson Labs) were fed with a high fat diet (HFD) for 12 weeks in the presence and absence of levan having β-[2,6]-glycosidic linkages. More specifically, the mice were fed either standard diet (SD; 5.8% fat, 44.3% carbohydrate, 19.1% protein) or high fat (HFD; 60% fat, 20% carbohydrate, 20% protein) diet for 12 weeks. Levan having β-[2,6]-glycosidic linkages was administered by gavage (250 mg/Kg) for the last 4 weeks of HFD feeding for a subgroup of mice, the experimental group. Bodyweight, blood glucose, body composition, and lipid profile were determined. Vascular function and endothelial function markers were studied in aorta and mesenteric resistance arteries (MRA). Furthermore, body weight was measured weekly, lean and fat mass were measured by EchoMRl, blood glucose and lipid plasma levels were measured, and the thoracic aorta and mesenteric resistance arteries (MRA) were harvested and used for western blot, quantitative real time (qRT-) PCR and vascular reactivity using the wire myograph.

Results, reported as means ± standard error (SE), were analyzed using GraphPad Prism 9 (GraphPad Software, San Diego, CA). T-tests and one-way ANOVA were used to compare certain paired parameters. Unless specified otherwise, the values for p are *p<0.05; **p<0.01; ***p<0.001; and ****p<0.0001.

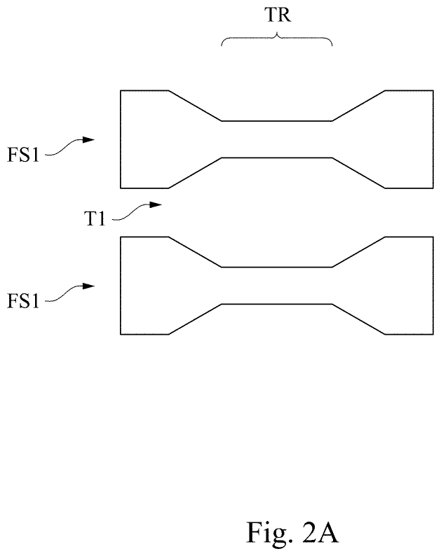

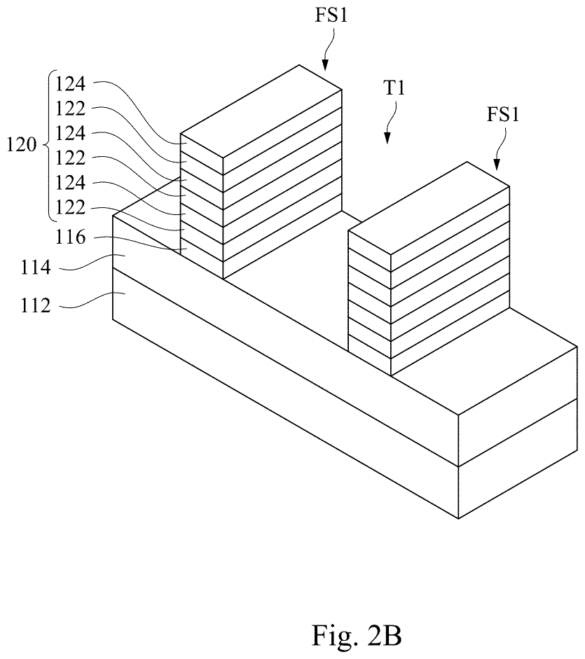

As shown in FIGS. 2A-2H, the administration of low molecular weight levan having β-[2,6]-glycosidic linkages decreased body weight, and blood glucose, reversed the body composition (% of fat and lean mass) and improved the lipid profile in mice with obesity. The body weight of the experimental mice having received low molecular weight levan having β-[2,6]-glycosidic linkages treatment decreased when compared to the HFD fed mice that did not receive the treatment (low molecular weight levan). The fat mass %, blood glucose, total cholesterol, LDL, HDL, and triglyceride were all also observed to be significantly decreased in the experimental mice compared to the untreated HFD fed mice, whereas the lean mass % was observed to be significantly increased.

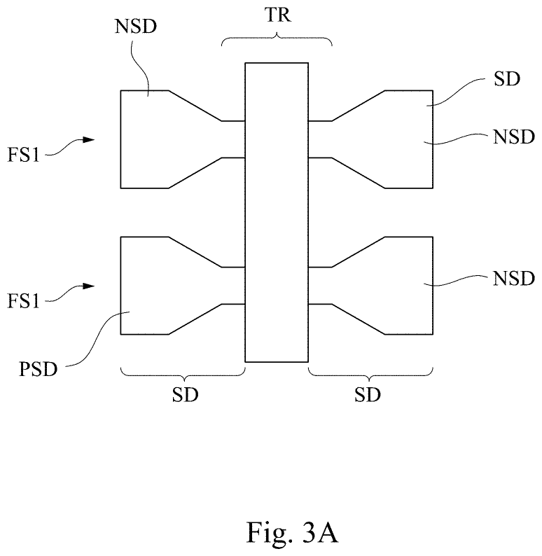

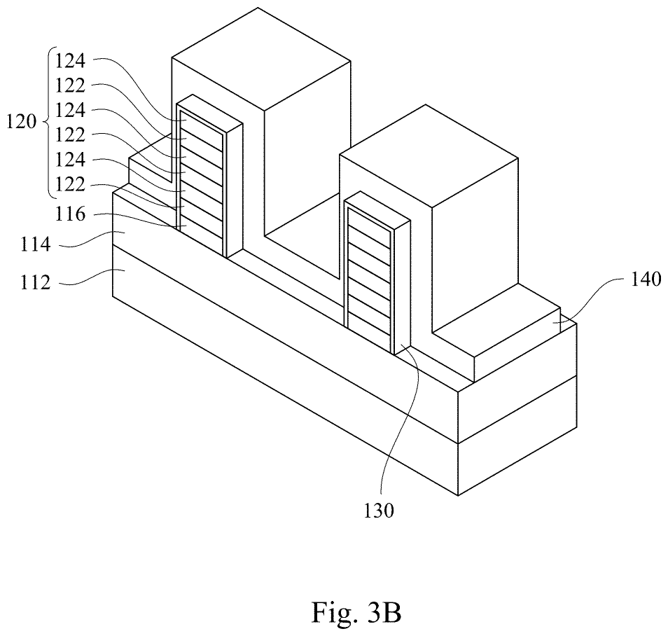

As shown in FIGS. 3A-3W the administration of low molecular levan having β-[2,6]-glycosidic linkages treatment improved vascular function in aorta from mice with obesity. This data showed that obesity damaged the conductance artery (thoracic aortic) endothelial function and treatment with low MW levan was able to preserve the endothelial function. Inflammation, oxidative stress as well as endoplasmic reticulum stress are key elements in exacerbating the endothelial dysfunction. FIGS. 3A-3W showed that all these parameters were elevated in obesity and treatment with low MW levan significantly reduced them.

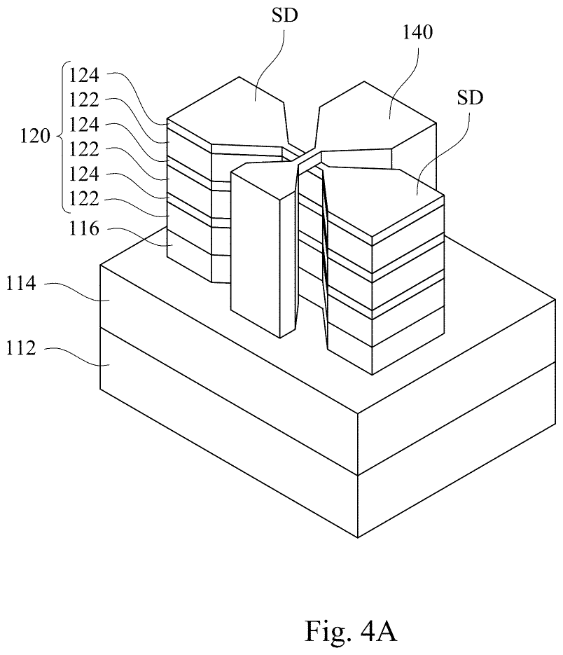

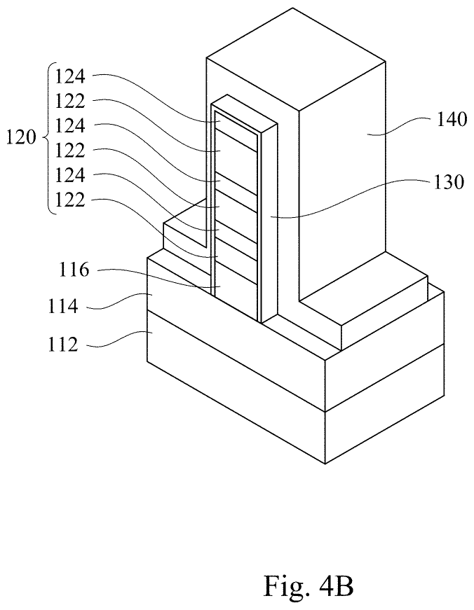

As shown in FIGS. 4A-4J the administration of low molecular levan having β-[2,6]-glycosidic linkages treatment improved vascular function in mesenteric arteries from mice with obesity. This data showed that obesity damaged the resistance arteries (mesenteric artery) endothelial function and treatment with low MW levan was able to preserve the endothelial function. FIGS. 4A-4J showed that inflammation, oxidative stress as well as endoplasmic reticulum stress were elevated in obesity and treatment with low levan significantly reduced them.

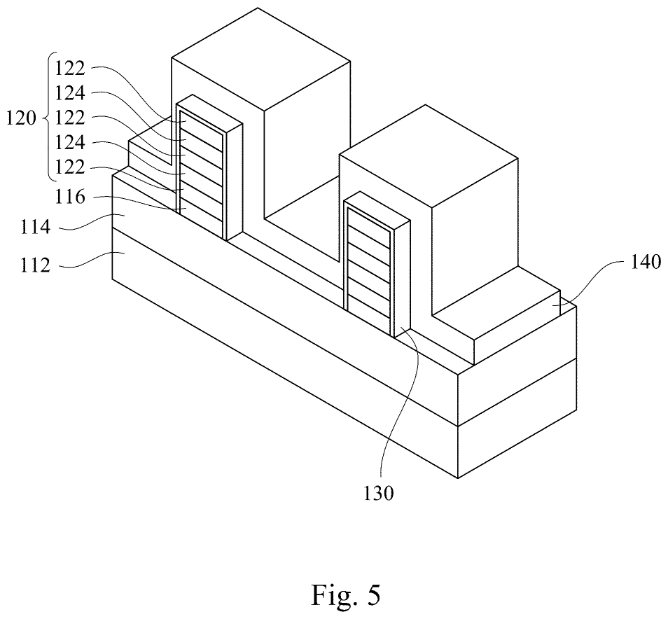

As shown in FIGS. 5A-5L the administration of low molecular levan having β-[2,6]-glycosidic linkages treatment reduced inflammation, oxidative stress and ER stress in proximal colon from mice with obesity. Increased inflammation, oxidative stress and ER stress in the proximal colon lead to gastrointestinal tract diseases that can affect the gut bacteria composition and the release of gut metabolites that play an important role in regulating the cardiovascular system. Reducing inflammation, oxidative stress and ER stress in the proximal colon by low MW levan as demonstrated in FIGS. 5A-5L, improved the gut function, bacteria composition and the release of the metabolites that can affect the cardiovascular system.

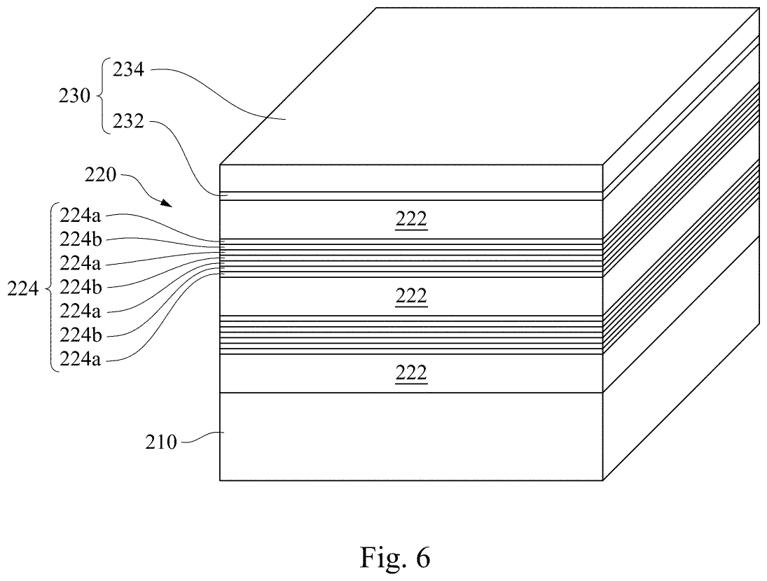

As shown in FIGS. 6A-6L the administration of low molecular levan having β-[2,6]-glycosidic linkages treatment reduced inflammation, oxidative stress and ER stress in distal colon from mice with obesity. Increased inflammation, oxidative stress and ER stress in the distal colon lead to gastrointestinal tract diseases that can affect the gut bacteria composition and the release of gut metabolites that play an important role in regulating the cardiovascular system. Reducing inflammation, oxidative stress and ER stress in the distal colon by low MW levan as demonstrated in FIGS. 6A-6L, improved the gut function, bacteria composition and the release of the metabolites that can affect the cardiovascular system.

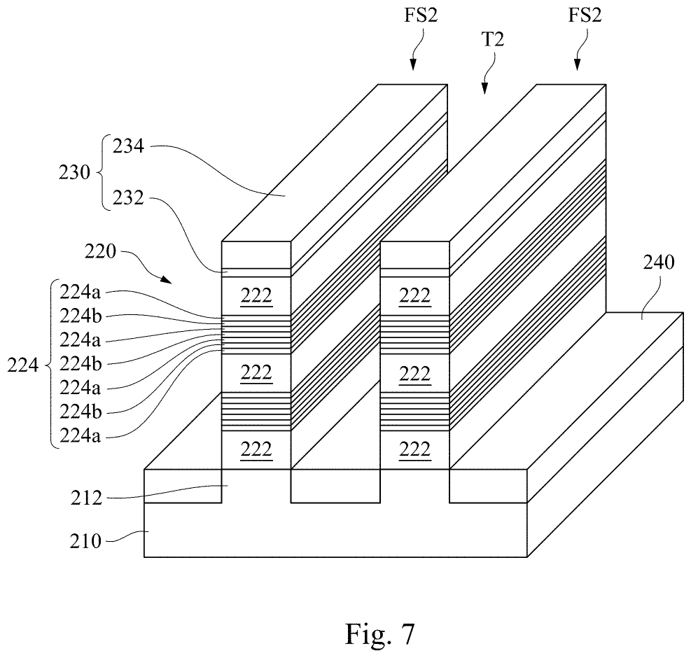

As shown in FIGS. 7A-7B the administration of low molecular levan having β-[2,6]-glycosidic linkages treatment improved liver function in mice with obesity. Due to fat accumulation, obesity can lead to liver dysfunction. Treatment with low MW levan as demonstrated in FIGS. 7A-7B showed an improvement of the liver function additionally, it showed that the dose used was not toxic to the mice.

Low MW levan β-[2,6]-glycosidic linkages treatment reduced the body weight in obese mice. Blood glucose, % of fat mass, total cholesterol, LDL levels were significantly decreased, and the % of lean mass was increased after low MW levan β-[2,6]-glycosidic linkages treatment. Aortic and MRA response to sodium nitroprusside (SNP) was similar among groups. However, the vascular response to acetylcholine (Ach) was improved in the treated group. This was associated with a decreased level of vascular endoplasmic reticulum stress, inflammation, and oxidative stress in thoracic aorta, mesenteric arteries, proximal colon and distal colon.

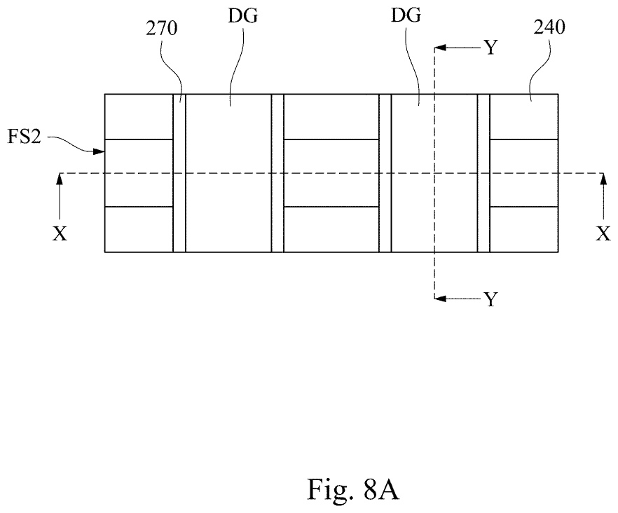

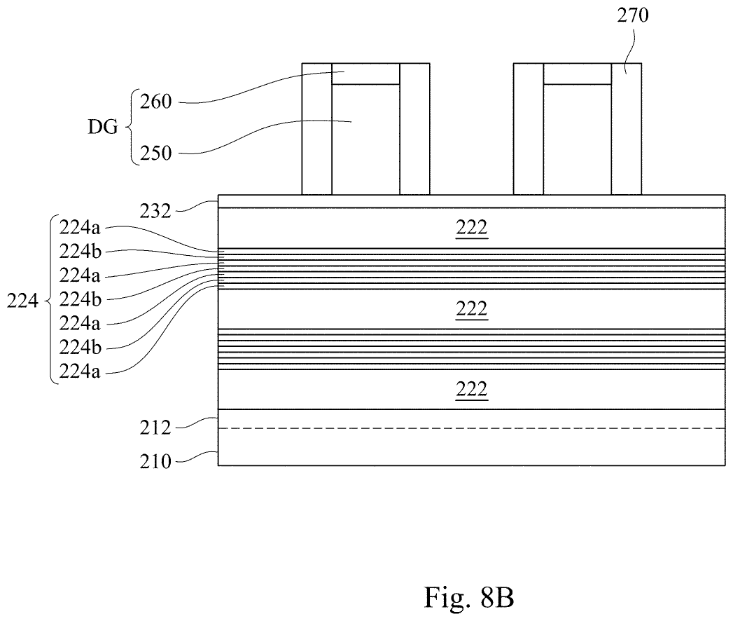

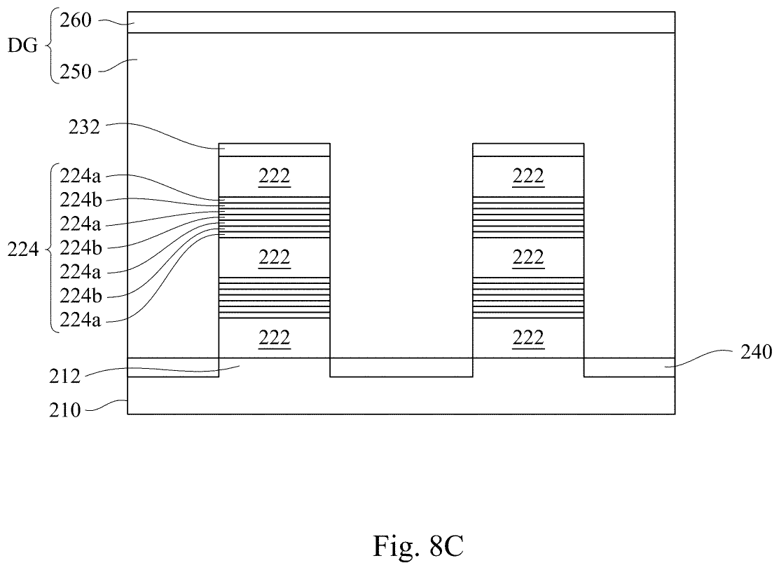

As shown in FIGS. 8A-8Z the administration of low molecular levan having β-[2,6]-glycosidic linkages treatment reduced blood pressure and improved vascular function in the aorta of mice with hypertension. The reduction of blood pressure by the low MW levan indicated that the low MW levan can regulate the blood pressure through the gut. The present treatment can therefore be used to replace antihypertensive drugs that have side effects.

An increase in vascular contraction and a decrease in vascular relaxation are cause/effect for hypertension. Treatment with low MW levan improved the vascular function by reducing the exacerbated vascular contraction and by improving the relaxation.

Inflammation, oxidative stress as well as endoplasmic reticulum stress are key elements in exacerbating the endothelial dysfunction. As shown in FIGS. 8A-8Z, all these parameters were elevated in hypertension and treatment with low MW levan significantly reduced them.

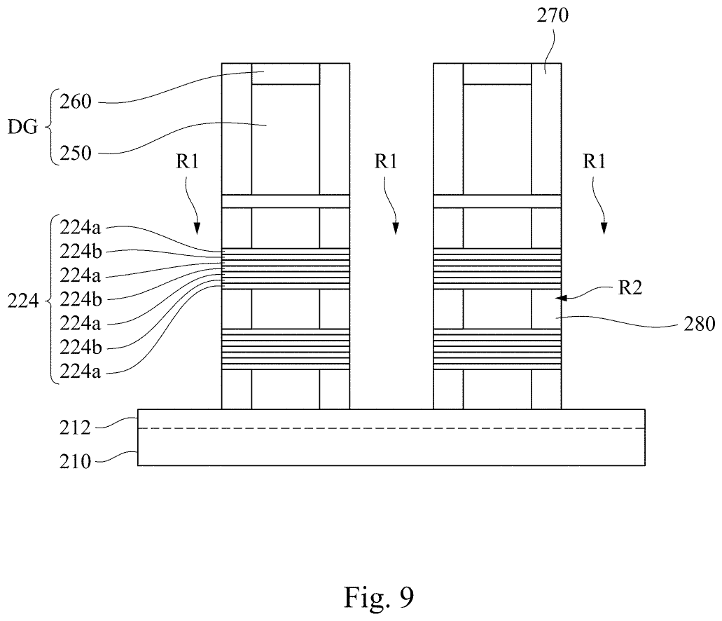

As shown in FIGS. 9A-9K the administration of low molecular levan having β-[2,6]-glycosidic linkages treatment reduced inflammation, oxidative stress and ER stress in the proximal colon of mice with hypertension. Increased inflammation, oxidative stress and ER stress in distal colon lead to gastrointestinal tract diseases that can affect the gut bacteria composition and the release of gut metabolites that play an important role in regulating the cardiovascular system. Reducing inflammation, oxidative stress and ER stress in the distal colon by low MW levan improved the gut function, bacteria composition and the release of the metabolites that can affect the cardiovascular system.

As shown in FIGS. 10A-10K the administration of low molecular levan having β-[2,6]-glycosidic linkages treatment reduced inflammation, oxidative stress and ER stress in distal colon from mice with hypertension. Increased inflammation, oxidative stress and ER stress in distal colon lead to gastrointestinal tract diseases that can affect the gut bacteria composition and the release of gut metabolites that play an important role in regulating the cardiovascular system. Reducing inflammation, oxidative stress and ER stress in the distal colon by low levan improved the gut function, bacteria composition and the release of the metabolites that can affect the cardiovascular system.

As shown in FIGS. 11A-11B the administration of low molecular levan having β-[2,6]-glycosidic linkages treatment did not cause damage to the liver function in hypertensive mice, indicating that the dose and treatment period was not toxic for the mice.

What is claimed is:

1. A levan having at least 85% of glycosidic linkages being β-[2,6]-glycosidic linkages.

2. The levan of claim 1, wherein the levan has a molecular weight of less than 20 kDa.

3. The levan of claim 1, wherein the levan has a molecular weight of from 5 to 20 kDa.

4. The levan of claim 1, wherein the levan has at least 90% of the glycosidic linkages being β-[2,6]-glycosidic linkages.

5. The levan of claim 1, wherein the levan has a terminal unit ratio of fructose to glucose of from 4 to 5.

6. A method of treating obesity and/or hypertension in a subject in need thereof comprising administering to the subject a therapeutically effective dose of levan.

7. The method of claim 6, wherein the levan has a molecular weight of less than 20 kDa.

8. The method of claim 6 wherein the levan has a molecular weight of from 5 to 20 kDa. The method of claim 6, wherein at least 85% of glycosidic linkages in the levan are β-[2,6]-glycosidic linkages.

9. The method of claim 6, wherein the levan has at least 90% of the glycosidic linkages being β-[2,6]-glycosidic linkages.

10. The method of claim 6, wherein the levan has a terminal unit ratio of fructose to glucose of from 4 to 5.

11. A method of treating obesity and/or hypertension in a subject in need thereof comprising administering to the subject a pharmaceutical composition comprising levan and a pharmaceutically acceptable excipient.

12. The method of claim 11, wherein the levan has a molecular weight of less than 20 kDa.

13. The method of claim 11 wherein the levan has a molecular weight of from 5 to 20 kDa.

14. The method of claim 11, wherein at least 85% of glycosidic linkages in the levan are β-[2,6]-glycosidic linkages.

15. The method of claim 11, wherein at least 90% of the glycosidic linkages in the levan are β-[2,6]-glycosidic linkages.

16. The method of claim 11, wherein the levan has a terminal unit ratio of fructose to glucose of from 4 to 5.

17. A pharmaceutical composition comprising the levan of claim 1 and a pharmaceutically acceptable carrier.
